(19) United States
(12) Patent Application Publication
(10) Pub. No.: US 2022/0226180 A1
(43) Pub. Date: Jul. 21, 2022

(54) Cart for Medical Equipment with Built-In Power Supply

(71) Applicant: Carr Innovations, LLC, Gallatin, TN (US)

(72) Inventors: Bradley E. Lawson, Gallatin, TN (US); Kevin A. Gregory, Gallatin, TN (US)

(21) Appl. No.: 17/579,799

(22) Filed: Jan. 20, 2022

Related U.S. Application Data
(60) Provisional application No. 63/139,603, filed on Jan. 20, 2021.

(51) Int. Cl. A61G 12/00; H02J 9/06

(52) U.S. Cl. CPC A61G 12/001; H02J 9/062; A61G 12/008

(57) ABSTRACT

An equipment cart for medical equipment, such as medical disinfection equipment, includes a base with a support surface for securing the equipment and casters that allow the equipment cart to be moved. A mechanical locking mechanism for the equipment may be provided. The equipment cart has an upright support connected to the base, and a work surface supported by the upright support and spaced from the base. A power system, including an uninterruptible power supply, is provided onboard the equipment cart and supplies AC power to one or more electrical outlets on the equipment cart in the form of generally sinusoidal AC power.

### CROSS-REFERENCE TO RELATED APPLICATIONS

This application claims priority to, and the benefit of, U.S. Provisional Patent Application No. 63/139,603, filed Jan. 20, 2021. The contents of that application are incorporated by reference herein in their entirety.

### TECHNICAL FIELD

Generally speaking, the invention relates to medical equipment, and more particularly, to carts and power supplies suitable for medical equipment.

### BACKGROUND

Many pieces of medical equipment are designed to be used with multiple patients. This is particularly true of expensive electronic diagnostic equipment, like ultrasound probes. While disposable covers and other kinds of disposable barriers are often used to minimize some types of equipment contamination where and when possible, equipment still gets contaminated, especially if it must enter a body cavity in normal use.

Procedures for decontaminating equipment vary widely according to the type of equipment and the types of harsh exposure that the equipment can sustain without damage. For example, handheld metal medical and surgical tools are often autoclaved for high temperature sterilization. Medical devices that include plastic components, or other components that cannot withstand high temperatures, are often chemically disinfected or, in some cases, disinfected by exposure to UV light.

The TROPHON® 2 disinfection apparatus (Nanosonics Limited, Sydney, Australia) is one example of a disinfection apparatus that is particularly adapted for surface, transvaginal, and transrectal ultrasound probes. Within a generally rectilinear cabinet, the device uses a chemical disinfectant mist driven by ultrasonic vibration to achieve disinfection.

Many pieces of medical equipment are portable. Many pieces of decontamination equipment are not. This means that in most cases, contaminated equipment must be brought to the decontamination equipment. This can cause serious inconvenience for medical staff and an impediment to workflow. If the decontamination equipment can be moved, the means for doing so are often imperfect, and do not provide for all of the equipment's needs.

One of those needs is power. When working with many pieces of medical equipment and disinfection equipment, it is desirable to provide a continuous source of power. If a piece of equipment is turned off, it may be necessary to subject it to time-consuming start-up or recalibration procedures. Most pieces of medical equipment are supplied with alternating-current (AC) power from traditional wall outlets. Uninterruptable power supplies (UPS), which use batteries to store energy and circuitry to deliver that energy as high-voltage AC power, are also becoming more common.

Most UPS units are ill-suited for sensitive medical equipment. For example, most lower-end UPS units on the market provide a stepped or square-wave AC voltage waveform, instead of the pure sinusoidal waveform provided by the typical power grid. While some equipment can function using square-wave AC, square-wave UPS systems can also cause unreliability and equipment failure. However, there are very few UPS systems that are particularly adapted for medical equipment and can also facilitate equipment portability.

### BRIEF SUMMARY

One aspect of the invention relates to an equipment cart for medical and disinfection equipment. The equipment cart is designed to mount disinfection equipment in a position near the base of the cart and may include structure to fix the disinfection equipment in place. Casters are provided on the underside of the base to allow for movement of the equipment cart. A telescoping support post supports a work surface at a position above the base and disinfection equipment. The work surface may provide a fully-equipped disinfection workstation with a storage drawer and holders for disinfecting wipes, gloves, and pieces of equipment. Various locking mechanisms may be present, e.g., to lock the storage drawer, and to lock the piece of disinfection equipment to the base of the equipment cart. The equipment cart may have an electrical system to supply power to the disinfection equipment and other peripherals.

Another aspect of the invention relates to an electrical system for a medical equipment cart. The electrical system routes power from a high-voltage AC power source to the equipment when a high-voltage AC power source is available. The electrical system also includes a battery and battery charging circuit that are charged using the AC power source when it is available. When the AC power source is not available, the electrical system uses power from the battery to generate a pure AC sine wave, which is then stepped up to high voltage. The pure AC sine wave is generated by generating a first voltage signal and a second voltage signal and sending both voltage signals through a comparator. The first and second voltage signals may be, e.g., triangular waves, with the first signal being a higher-frequency “fast” wave and the second signal being a lower-frequency “slow” wave. The output of the comparator is further processed to generate the pure AC sine wave. For example, a half-wave rectifier and a pair of flip-flops may be used to generate the pure AC sine wave from the output of the comparator.

Other aspects, features, and advantages of the invention will be set forth in the description that follows.

### BRIEF DESCRIPTION OF THE DRAWING FIGURES

The invention will be described with respect to the following drawing figures, in which like numerals represent like features throughout the description, and in which:

FIG. 1 is a perspective view of an equipment cart according to one embodiment of the invention;

FIG. 2 is an exploded perspective view of the equipment cart of FIG. 1;

FIG. 3 is a side elevational view of the equipment cart of FIG. 1;

FIG. 4 is a rear elevational view of the equipment cart of FIG. 1;

FIG. 5 is a bottom view of the equipment cart of FIG. 1;

FIG. 6 is a perspective view of an equipment cart according to another embodiment of the invention;

FIG. 7 is a perspective view of the lower portion of an equipment cart according to embodiments of the invention, illustrating a locking mechanism for retaining equipment; and

FIGS. 8-1, 8-2, and 8-3 are each a portion of a circuit diagram of a power supply circuit that may be installed on an equipment cart in embodiments of the invention.

### DETAILED DESCRIPTION

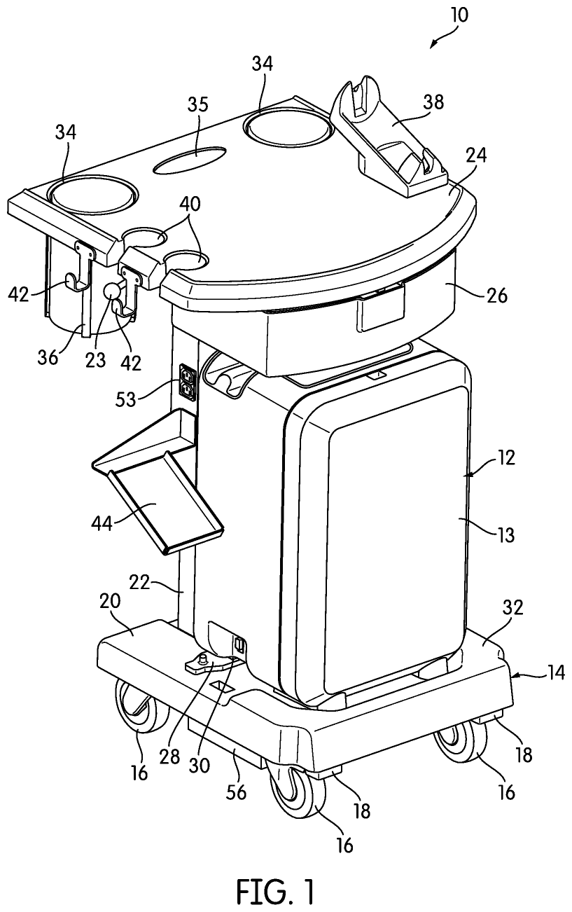

FIG. 1 is a perspective view of an equipment cart, generally indicated at 10, according to one embodiment of the invention. In FIG. 1, the equipment cart 10 is shown with a piece of disinfection equipment 12 installed. The disinfection equipment 12 of FIG. 1 has the size and proportions of the TROPHON® 2 device described above, and may be the TROPHON® 2 device described above, although any disinfection equipment 12 may be mounted on the equipment cart 10.

The equipment cart 10 is intended to serve as a mobile disinfection workstation, with a base 14 that includes casters 16 for movement. In the illustrated embodiment, there are four casters 16, positioned in a rectangular layout supporting the base 14, although any number of casters 16 may be used, so long as that number of casters 16 provides for a stable, moveable base 14. The front casters 16 each have a standard frictional braking mechanism, the actuation levers 18 for which can be seen in FIG. 1. The braking mechanism allows the equipment cart 10 to be locked into place once a desired location has been reached. In other embodiments, other forms of wheels may be used to make the equipment cart moveable.

The base 14 includes a lower shelf 20, on which the disinfection equipment 12 rests. A single telescoping support post 22 rises from the base 14 to provide support for the other elements of the equipment cart. At its upper extent, a work surface 24 is cantilevered from the support post 22. A drawer 26 lies under the work surface 24.

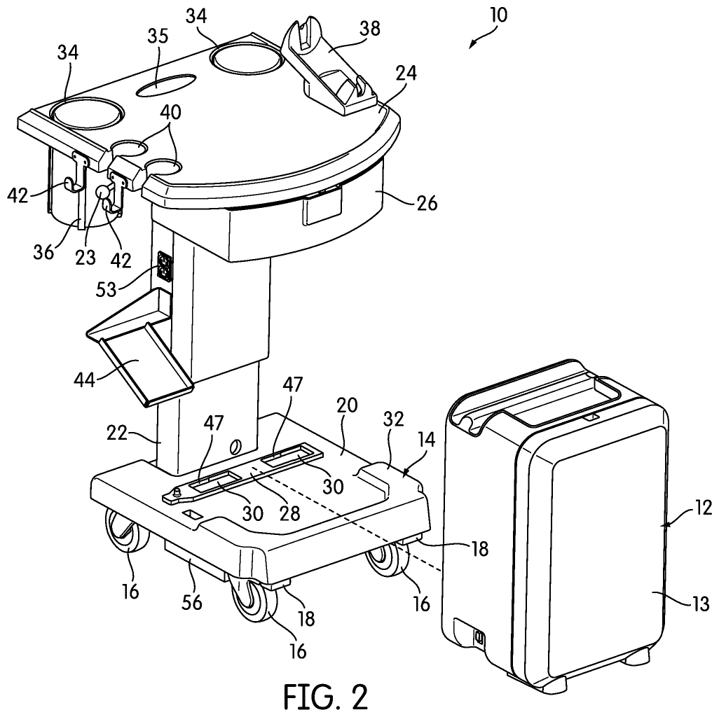

FIG. 2 is an exploded perspective view illustrating the equipment cart 10 with the disinfection equipment 12 exploded out, in order to show the details of the lower shelf 20. The lower shelf 20 may be of any size that is appropriate for the disinfection equipment 12.

One particular advantage of the equipment cart 10 is that by mounting the disinfection equipment on a low shelf, like the lower shelf 20, the weight of the disinfection equipment 12 is less likely to tip the equipment cart 10. Low mounting also means that the equipment cart 10 can include a workspace, like the work surface 24, at the appropriate height for a user.

As shown in the view of FIG. 2, the lower shelf 20 may have structure on it to secure the disinfection equipment 12. That structure may vary from embodiment to embodiment. In some cases, the disinfection equipment 12 may be rigidly clamped to the lower shelf 20, while in other cases, the structure may be limited to depressions or openings into which the feet or lower portions of the disinfection equipment 12 fit. In the illustrated embodiment, a relatively thick plate 28 is secured to the lower shelf 20. The bracket 28 has openings 30 in which the feet from the disinfection equipment 12 rest, and forms a locking mechanism for the disinfection equipment 12, as will be described below in more detail. The forward portion of the lower shelf 20 also forms a berm 32 that helps to prevent the disinfection equipment 12 from shifting out of place when the equipment cart 10 is moved.

The orientation of the disinfection equipment 12 on the cart, and the manner in which it opens and is used, may affect the arrangement of the equipment cart 10 and its base 14 and lower shelf 20. In this embodiment, the disinfection equipment 12 has the form of a cabinet with a hinged door 13 that swings open outwardly. Thus, the height of the berm 32 at the front of the base 14 is sufficient to help retain the disinfection equipment 12, but not so high as to obstruct the movement of the door 32.

With the disinfection equipment 12 exploded away, other details of the equipment cart 10 can be seen in the view of FIG. 2. For example, in the illustrated embodiment, the support post 22 has a rectangular cross-sectional shape with a larger width than its depth. In this embodiment, the support post 22 is in two sections, although more sections may be used, depending on the minimum and maximum heights that are to be used. The telescoping mechanism itself may be of any type. For example, a pneumatic height adjustment mechanism may be used. The height adjustment lever 23 for such a mechanism may be positioned just under the work surface 20, where it can be easily actuated.

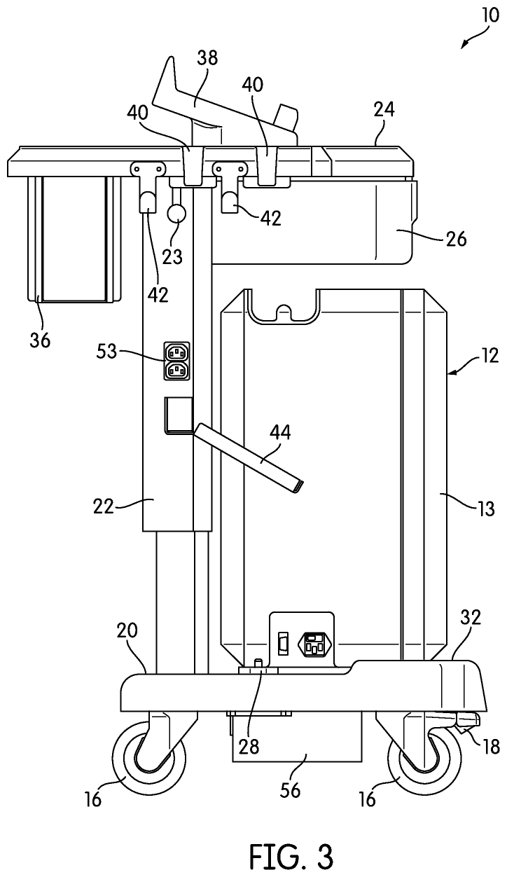

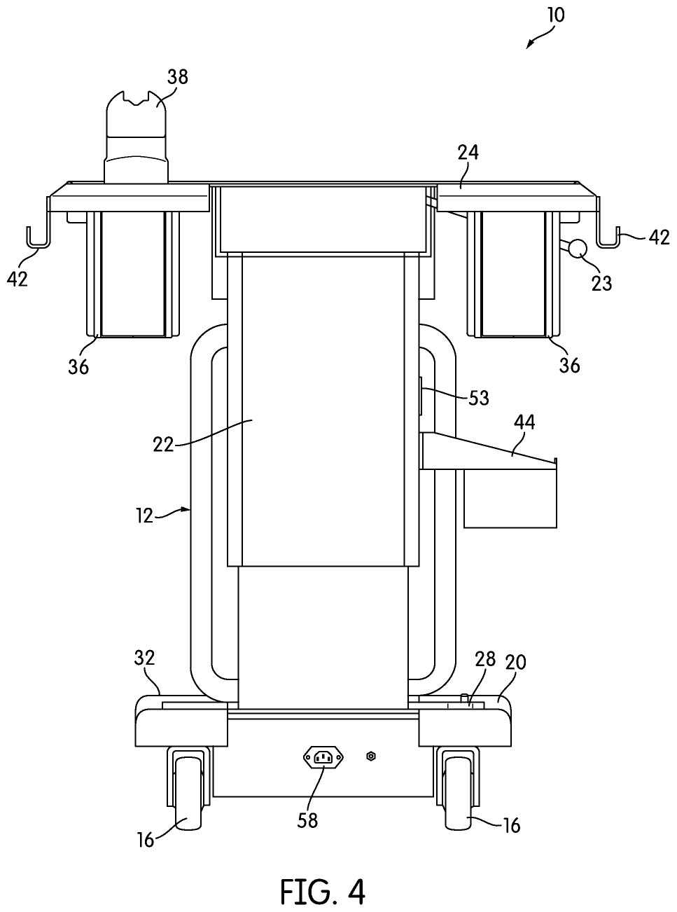

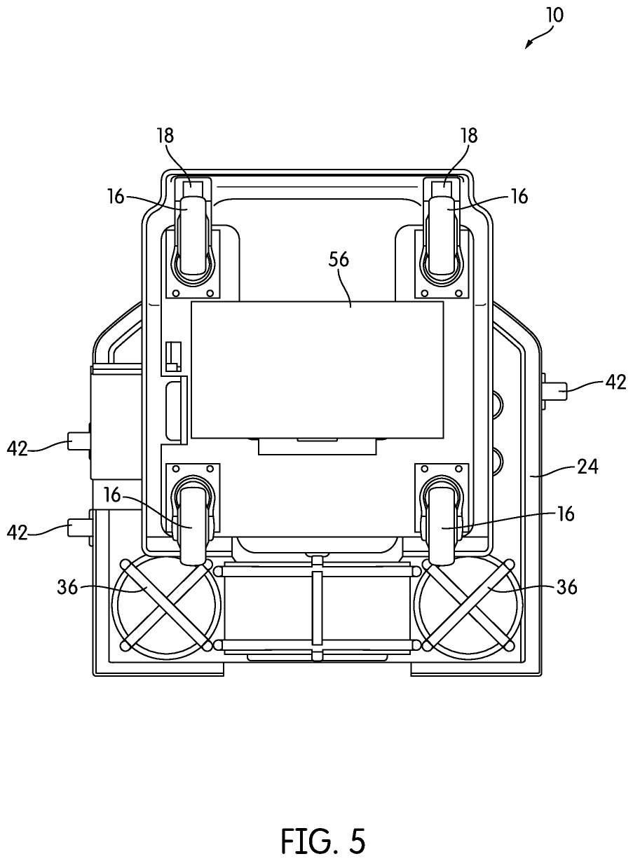

FIG. 3 is a side elevational view of the equipment cart 10, FIG. 4 is a rear elevational view, and FIG. 5 is a bottom plan view. As can be seen in these figures, the equipment cart 10 of the illustrated embodiment is configured to be used as a cleaning workstation, above and beyond the presence of the disinfection equipment 12 itself. The rear corners of the work surface 24 have circular openings 34 that open into cylindrical brackets 36. These openings 34 and brackets 36 are shaped and sized for the cylindrical cannisters common to disinfecting wet wipes. A glove dispenser 35, sized for a box of gloves, is also provided in the rear portion of the work surface 24, between the pair of openings and their associated brackets 36. A probe holder 38 on one side of the work surface 24 is sized to hold a handheld ultrasound probe for cleaning. A set of circular cut-out openings 40 along the opposite side of the work surface 24 are provided to hold equipment, as are hooks 42 on the lateral sides of the work surface 24. Below the work surface 24, a small shelf-holder 44 extends from the upper portion of the support post 22. The shelf-holder 44 may, for example, be used to hold a printer, such as a label printer.

Thus, fully equipped, the equipment cart 10 may serve as a workstation that has all necessary equipment to remove gross soil and contamination from a piece of medical equipment before it is placed in the disinfecting equipment. A laptop and printer may be installed on the work surface 24 and the shelf-holder 44 in order to keep disinfection or other related compliance records.

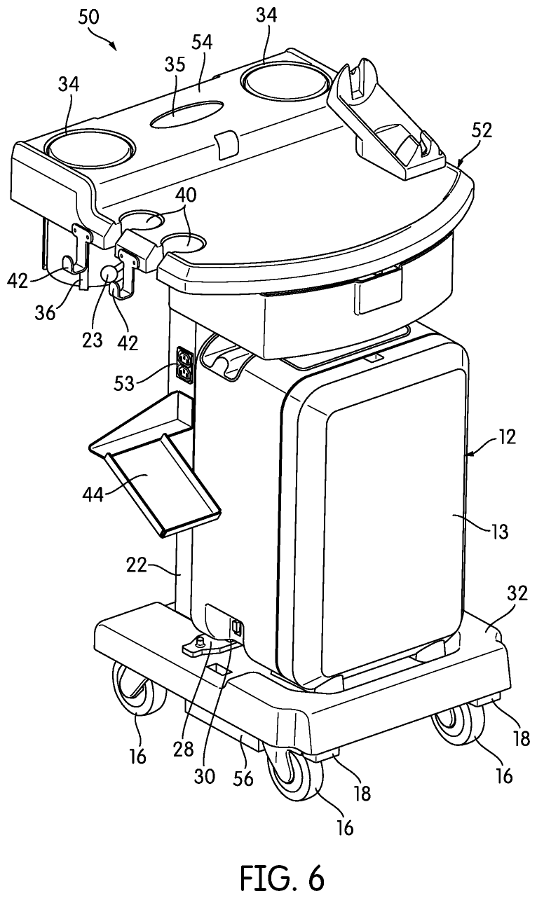

This particular configuration of the equipment cart 10 is not the only possible configuration. FIG. 6, for example, is a perspective view of another equipment cart, generally indicated at 50. The equipment cart 50 is generally identical to the equipment cart 10 described above, except for the configuration of its work surface 52. The work surface 52 of the equipment cart 50 is stepped, such that the rear area 54 of the work surface 52 is raised.

Both equipment carts 10, 50 include locks and security measures. Chemical disinfection may involve chemicals that are toxic, corrosive, strong oxidizers, or are otherwise hazardous. For example, chemicals like 35% hydrogen peroxide are common. Because of this, it is helpful if the equipment cart 10, 50 has some locks. For example, the drawer 26 may be locked, either with a key lock mechanism, or with a proximity locking mechanism, like a radio-frequency identification (RFID) locking mechanism. An RFID locking mechanism uses a low-power radio-frequency transmitter to interrogate a nearby RF element, which may be either powered or unpowered. If the nearby element transmits the correct identifier, the locking mechanism unlocks.

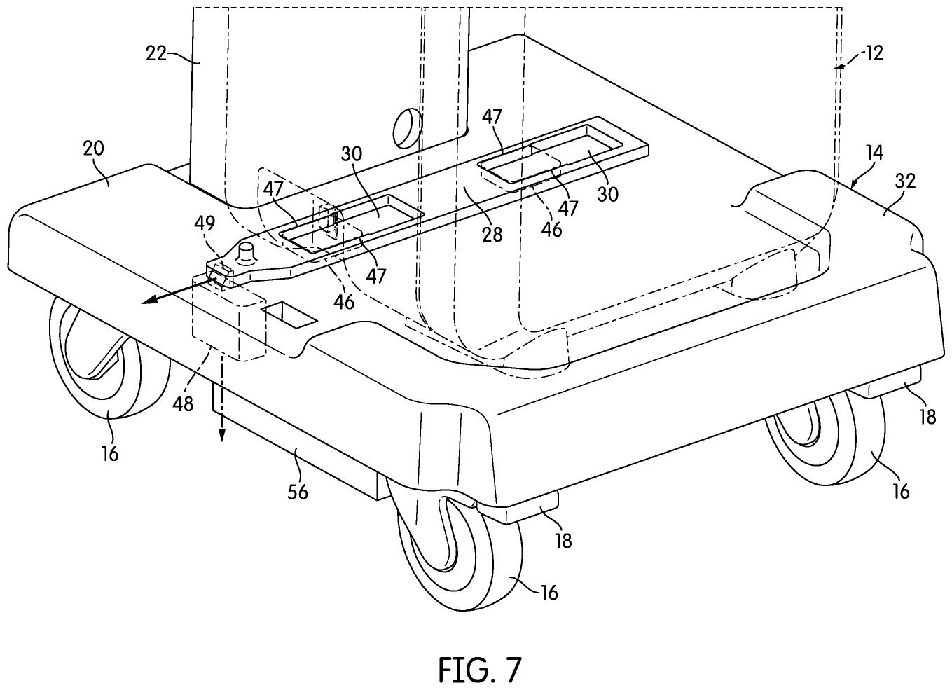

In addition to securing peripherals and chemicals within the drawer 26, locking mechanisms may be present elsewhere as well. FIG. 7 is a perspective view of the base 14 of the equipment cart 10, 50, with the disinfection equipment 12 shown in phantom lines. As was described above with respect to FIG. 2, there is a plate 28 on the lower shelf 20 of the base 14. That plate 28 engages with the feet 46 of the disinfection equipment 12 and serves as a locking mechanism. More specifically, the openings 30 in the plate 28 have two sections. With respect to the coordinate system of FIG. 7, on the right side of each opening 30, the walls of the opening 30 are straight-sided. On the left side of each opening, the long walls have a section 47 with walls that are canted inward. The inward cant of the left section 47 of the opening 30 matches a cant of the feet 46, meaning that in the position shown in FIG. 7, the disinfection equipment is positively engaged by the plate 28. In order to release the engagement, it is necessary to slide the plate 28 to the left, so that the feet 46 are no longer engaged by the canted sections 47 of the openings. However, another locking mechanism 48 is mounted within the base 14, with an upwardly projecting bolt 49 that engages a complementary opening in the plate 28 to lock it in place. When the bolt 49 is in the position shown in FIG. 7, the plate 28 cannot be slid rightward to disengage the canted sections 47 from the feet 46 of the disinfection equipment 12; thus, the disinfection equipment 12 cannot be lifted from the base 14. When the locking mechanism 48 is disengaged and the bolt 49 is withdrawn, the plate 28 can be slid rightward, freeing the disinfection equipment. The locking mechanism 48 may be a key-actuated locking mechanism, an RFID locking mechanism, or a locking mechanism that is actuated in some other way. In addition to preventing the unauthorized removal or theft of the disinfection equipment 12 from the equipment cart 10, 50, the plate 28 and its locking mechanism 48 also serve to secure the disinfection equipment 12 during movement of the equipment cart 10, 50 and against earthquake and other hazards.

The plate 28 and its openings 30 take advantage of an existing cant to the feet 46 of the disinfection equipment 12. As those of skill in the art will realize, many kinds of cooperating engaging features may be used to lock a structure such as the plate 28 to a piece of disinfection equipment.

The equipment carts 10, 50 have their own onboard electrical systems. As can be seen in FIGS. 1-6, electrical outlets 55 are provided on one side of the support post 22 to supply power to the disinfection equipment 12 and to other peripherals that may be used with the equipment carts 10, 50. The main components of the electrical system are contained in a housing 56 that is mounted on the underside of the lower shelf 20. The housing 56 is electrically connected to an outlet 58 at the lower rear of the equipment cart 10, 50. The outlet 58 serves as a connector to plug the equipment cart 10, 50 into AC mains (i.e., building) power.

FIGS. 8-1, 8-2, and 8-3 are circuit diagrams of the main circuit, generally indicated at 100, of the equipment carts 10, 50, each figure showing a portion of the main circuit 100. For purposes of description, it may be assumed that the circuit illustrated in FIGS. 8-1 through 8-3 resides in the housing 56, although portions of the circuit may reside in other physical locations.

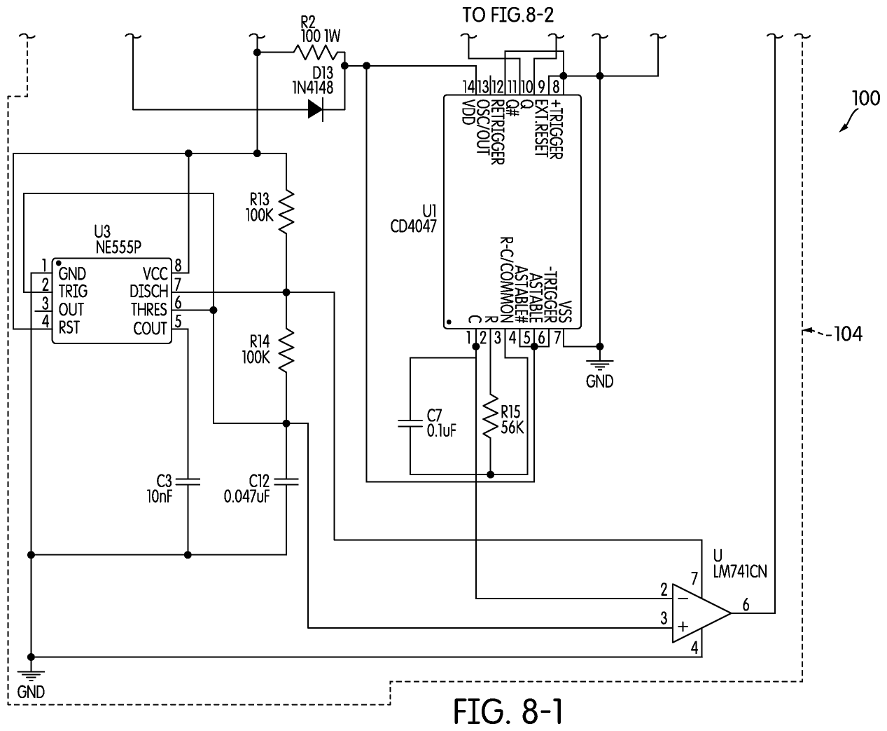

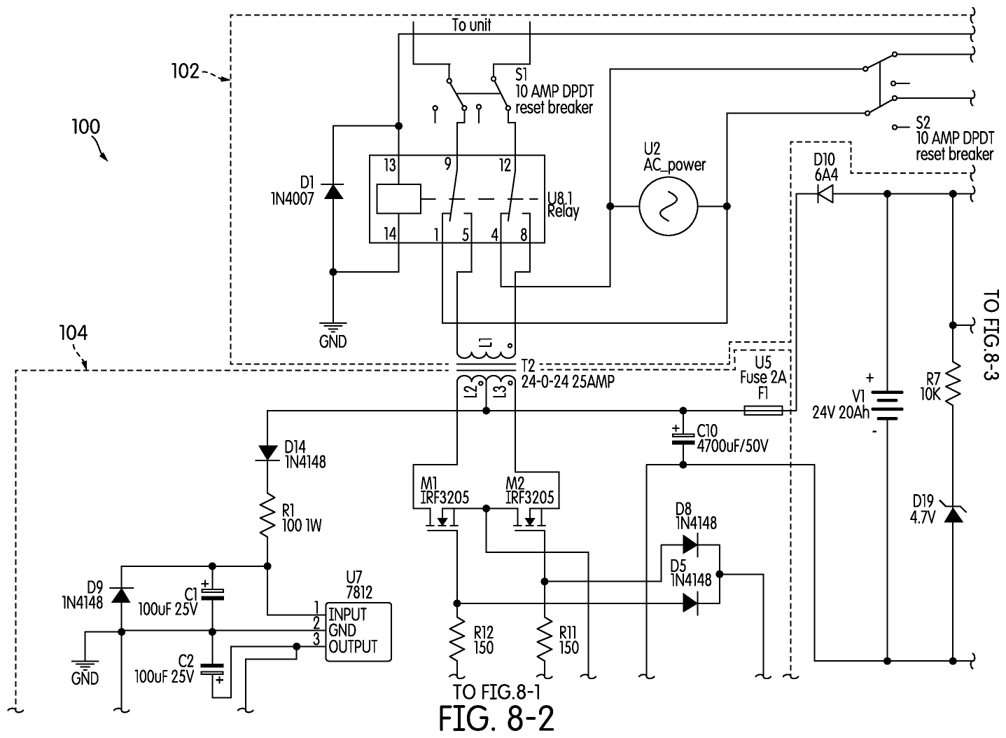

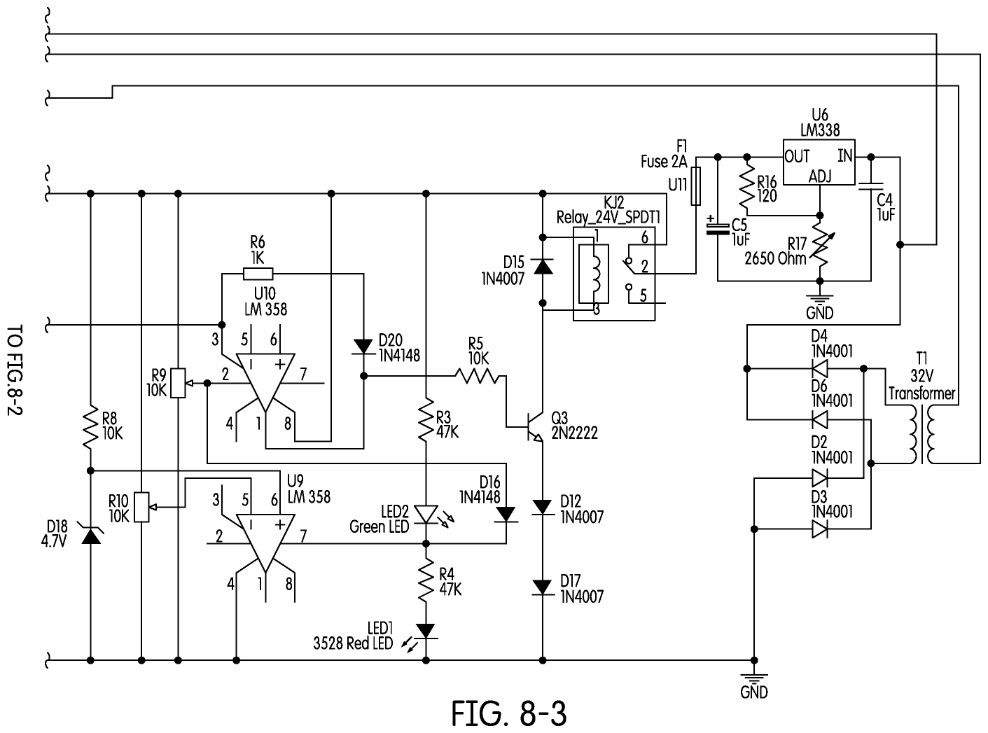

The main circuit 100 provides AC power to the electrical outlets 54 with a pure AC sine wave, even when it is not connected to internal power. To do this, it includes both high-voltage portions and low-voltage portions. (While the definition of “high voltage” varies according to the authority one consults, for purposes of this description, the term will refer to voltages over 50V.) Much of the high-voltage portion 102 of the circuit 100 is shown in FIG. 8-2.

The main circuit 100 illustrated in FIGS. 8-1 through 8-3 supplies power to the disinfection equipment 12 and the other equipment associated with the equipment cart 10, 50 from AC mains power when AC mains power is available, and from a battery when AC mains power is not available. Switching between these two power supplies will consume some short interval of time; thus, the main circuit 100 of FIGS. 8-1 through 8-3 may not be considered a “full” or “traditional” UPS in some contexts. In other embodiments, a power circuit may supply power entirely through a battery, using AC mains power, when available, to charge the battery. Thus, for purposes of this description, the term “UPS” should be read to include power circuits that switch between a battery and another power source, as well as power circuits that always supply power through a battery, but use another power source, if one is available, to maintain the battery's charge.

In the main circuit 100 of FIG. 8-2, switch 51 in FIG. 8-2 is a high-voltage DPDT breaker that supplies power to the power outlets 54 directly from AC mains power when AC mains power is available. As can be seen from the diagram of the high-voltage portion 102, the high-voltage portion 102 of this embodiment does not have particular power conditioning components (e.g., filters), although it may be provided with such components in other embodiments.

The switch S1 is connected to a relay U8.1 that connects either to the AC power, indicated as U2 in FIG. 8-2, or to a transformer T2 connected to the output of the signal generation portion 104 of the circuit 100. Thus, the relay U8.1 allows the power outlets 54 to be fed either directly by AC power or by battery-driven pure AC sine wave generated by the signal generation portion 104.

Power from the AC power U2 in FIG. 8-1 is routed to transformer T1 in FIG. 8-3, which, in this embodiment, is a 32V transformer that steps the power down from 120 VAC to 32 VAC. On the low-voltage side, power from the transformer T1 is fed to diodes D2, D3, D4, D6, which are in a full-bridge rectifier configuration and rectify the 32V AC power into 32V DC. The remainder of the circuit 100 shown in FIG. 8-3 is a battery charger for the 24V battery V1 (FIG. 8-2) that supplies power when the circuit 100 is not connected to external AC power. This battery charge portion 106 of the circuit uses an LM358 dual op-amp integrated circuit (IC) U9, U10 as comparators to monitor the voltage of the battery V1 during charge by sensing high-voltage (i.e., 28.8V) and low-voltage (i.e., 21.6V) conditions. When the voltage has reached the desired charge voltage, indicating that the battery V1 is fully charged, a voltage applied by the op amp U10 to the gate of the transistor Q3 cuts off the charge.

As those of skill in the art will realize, a full diagram of the battery charge portion 106 is included in FIG. 8-3 only for the sake of completeness; any suitable battery charging circuit may be used in embodiments of the invention.

As was noted briefly above, when the circuit 100 is not drawing power from AC mains, it draws from the battery V1 and modulates that power into a pure AC sine wave within the low-voltage signal generation portion 104 of the circuit 100. The low-voltage pure AC sine wave is then stepped up from low voltage to high voltage by the transformer T2.

Much of the low-voltage signal generation portion 104 is shown in FIG. 8-1, with the remainder in FIG. 8-2. Conceptually, the low-voltage signal generation portion 104 achieves its task by generating two waveforms of different characteristics and using those two waveforms as inputs to an op amp configured as a comparator. The output from the op-amp is used to create a series of half-waves, which is sent to a pair of flip-flops that generate the fully alternating sine wave from the half waves.

More specifically, the two waveforms of different characteristics are triangular or sawtooth-type waveforms in this embodiment. The first of the two sawtooth-type waveforms is generated by an NE555P timer IC U3. The connection of the 555 timer IC U3 with the resistors R13, R14 and capacitor C12 places the 555 timer IC in an astable configuration, allowing it to act as an oscillator. The voltage across the capacitor C12 is a triangular or sawtooth waveform in this configuration, and that waveform is sent to the noninverting input of an LM741CN op amp U.

A broader, “slower” triangular waveform is generated by a CD4047 multivibrator IC U1 in astable free-running operating mode. This output is connected to the inverting input of the LM741CN op amp U.

As was described briefly above, the output from the op amp U is first sent to two diodes D5, D8 in a half-wave rectifier configuration. The output of those diodes is sent to two IRF3205 flip-flops M1, M2, shown in FIG. 8-2, one set high and one set low, that produce a full sinusoid from the half-wave output of the diodes D5, D8. That sinusoid, still at low voltage, is stepped-up by the transformer T2 to high voltage, as was described above.

Unless otherwise noted, all electronic components in the circuit 100 are manufactured by, or can be obtained from, Texas Instruments, Inc. (Dallas, Tex., United States). As those of skill in the art will understand, the topology and components shown in FIGS. 8-1, 8-2, and 8-3 are only one way to implement a UPS with a “pure” sine wave power output. Variations and other approaches are possible.

While the invention has been described with respect to certain embodiments, the description is intended to be exemplary, rather than limiting. Modifications and changes may be made within the scope of the invention, which is defined by the appended claims.

What is claimed is:

1. An equipment cart, comprising:
a base having
a support surface,
an equipment lock coupled to the support surface, and
casters arranged to movably support the base;
an upright support attached to the base;
a work surface attached to the upright support, the work surface extending generally parallel to, and vertically spaced from, the support surface of the base; and
an electrical system disposed within the equipment cart, the electrical system including
a power input,
an uninterruptible power supply (UPS) coupled to the power input, the UPS including
one or more batteries, and
a circuit that produces an at least generally sinusoidal alternating current (AC) power output, and
being adapted to draw power from either or both of the power input and the one or more batteries; and
one or more AC power outlets coupled to the UPS.

2. The equipment cart of claim 1, wherein the equipment lock comprises:
a plate slideably mounted on the support surface, the plate having a first opening with a first portion and a second portion, the second portion of the plate having complementary engaging structure adapted to engage a portion of a piece of equipment; and
a lock mounted in association with the support surface and the plate, the lock adapted to retain the plate in a fixed position.

3. The equipment cart of claim 2, wherein walls of the second portion of the plate are angled.

4. The equipment cart of claim 2, wherein the lock comprises a moveable bolt positioned and adapted to engage with a complementary slot in the plate.

5. The equipment cart of claim 1, wherein the UPS comprises a switch that switches the one or more AC power outlets between the power input and the one or more batteries.

6. The equipment cart of claim 1, wherein the UPS comprises a high-voltage side and a low-voltage side with a transformer interposed therebetween.

7. The equipment cart of claim 6, wherein the low-voltage side of the UPS comprises:
a first oscillator generating a first non-sinusoidal waveform;
a second oscillator generating a second non-sinusoidal waveform, the second non-sinusoidal waveform having characteristics different from the first non-sinusoidal waveform; and
a comparator receiving the first non-sinusoidal waveform and the second non-sinusoidal waveform.

8. The equipment cart of claim 7, wherein the first non-sinusoidal waveform comprises a triangular or sawtooth waveform.

9. The equipment cart of claim 7, wherein the low-voltage side of the UPS comprises a half-wave rectifier connected to an output of the comparator.

10. The equipment cart of claim 9, wherein the low-voltage side of the UPS comprises a pair of flip-flops connected to an output of the half-wave rectifier, one of the pair of flip-flops set high and the other of the pair of flip-flops set low.

11. The equipment cart of claim 1, wherein the UPS is mounted to the base.

12. The equipment cart of claim 10, wherein the UPS is mounted to an underside of the support surface of the base.

13. The equipment cart of claim 1, further comprising a drawer mounted to an underside of the work surface.

14. The equipment cart of claim 12, wherein the drawer includes a lock.

15. The equipment cart of claim 1, wherein the work surface includes a step that divides the work surface into an upper work surface and a lower work surface.

16. The equipment cart of claim 1, wherein the work surface includes one or more built-in storage elements.

17. The equipment cart of claim 1, wherein the upright support comprises two or more portions arranged in telescoping fashion relative to one another.

18. The equipment cart of claim 1, wherein the base has a berm on a forward portion thereof.

19. The equipment cart of claim 1, wherein the equipment cart comprises a powered mobile disinfection workstation for medical equipment.
